(19) United States
(12) Patent Application Publication
(10) Pub. No.: US 2004/0231056 A1
(43) Pub. Date: Nov. 25, 2004

(54) Seating and/or reclining furniture and mattress with light-emitting means

(76) Inventor: Klaus Jansen, Buxtehude (DE)

Correspondence Address: TECHNOPROP COLTON, L.L.C., P O BOX 567685, ATLANTA, GA, 311567685

(21) Appl. No.: 10/483,040

(22) PCT Filed: Jul. 5, 2002
(86) PCT No.: PCT/EP02/07496

(30) Foreign Application Priority Data
Jul. 6, 2001 (DE) 201 11 076.8
Aug. 11, 2001 (DE) 201 13 383.0

(51) Int. Cl. A61N 5/06

(52) U.S. Cl. 5/600; 5/690; 607/100

(57) ABSTRACT

The invention relates to a piece of seating and/or reclining furniture, especially a bed, which emits rays of light. In particular, a mattress (10) is provided with illuminants (12) in the mattress core (17) and/or in the cover (18), or between the mattress core (17) and the cover (18). The light irradiation, to which a person (11) is exposed promotes the stimulus for sleeping and/or waking up. The mattress also promotes therapeutic effects, for example wound-healing properties.

### STATEMENT OF RELATED APPLICATIONS

This is the United States Patent Cooperation Treaty (PCT) Chapter II National Phase of PCT/EP02.07496 having an international filing date of 5 Jul. 2002, which claims priority on German patent application no. 20111076.8 having a filing date of 6 Jul. 2001 and German patent application no. 20113383.0 having a filing date of 11 Aug. 2001.

### BACKGROUND OF THE INVENTION

1. Technical Field

The invention relates to a mattress having at least one mattress core and a cover. Furthermore, the invention relates to a piece of seating and/or reclining furniture, in particular bed, with a frame and at least one mattress arranged thereon.

2. Prior Art

It is known from the field of chronobiology that light is the most significant factor influencing the inner circadian rhythm of human biological activity. Scientific studies have shown that light of preferably blue/green or white color, regardless of its intensity, triggers a more or less pronounced stimulus for waking up in persons exposed to such light. On the other hand, light preferably composed of red color tends to promote human relaxation, particularly in the evening hours, which may have a positive effect in stimulating sleep. Furthermore, it has been demonstrated that the described effects can also arise when the light is not sensed through the eyes but when other parts of the body are exposed to the light.

Light-permeable reclining surfaces are already known, for example in the form of a transparent supporting surface with air chambers (DE 37 19 346 A1) under which illuminants are arranged for the purpose of illuminating a person lying on the recliner. Although such disclosed illuminated reclining surfaces ensure an irradiation of light onto the person lying thereon, the reclining comfort is negligible when compared to that of a conventional piece of seating or reclining furniture. A further disadvantage of known light-permeable reclining surfaces is evident in the fact that the illuminants are arranged under the reclining surface, thereby weakening the light rays striking the top surface of the body. Finally, the known illuminated recliners can only be employed in conjunction with a specially constructed piece of reclining furniture.

### BRIEF SUMMARY OF THE INVENTION

The object of the invention is to provide a mattress and a piece of seating and/or reclining furniture that makes it possible to deliver intense light irradiation on the person lying thereon without any sacrifice of seating or lying comfort and with no need for a special substructure.

A mattress for realizing this object is characterized by illuminants arranged in the mattress core and/or in the cover, or between the mattress core and the cover. By virtue of having illuminants arranged in the mattress core and/or in the cover, or between the mattress core and the cover, it is possible to achieve effective light irradiation of the person lying on the mattress, but without sacrificing any seating or reclining comfort as compared to that offered by conventional mattresses. Furthermore, the mattress according to the invention corresponds in appearance to conventional mattresses, thus making it possible to use the mattress in conjunction with common furniture constructions. Such a mattress is suitable for reclining furniture, such as beds, as well as for seating furniture.

A preferred embodiment of the mattress according to the invention is furthermore characterized in that it has, preferably at its top side, at least one depression in which preferably a plurality of illuminants are arranged. The illuminants thus become an integrated component of the mattress core in that they can be completely housed in said depression. It is conceivable to have the depression designed as an oval hollow, a grooved section and/or as a continuous groove.

Pursuant to a further development of the mattress according to the invention, the depressions have an essentially T-shaped cross-section, with a narrow section of the depression proceeding from the top side of the mattress core. This design feature ensures that the illuminants within the mattress core are securely anchored without any additional securing means.

Pursuant to a further development of the mattress, each depression is aligned perpendicular to the longitudinal axis of the mattress and corresponds essentially to the width of the mattress core. This design feature ensures that illumination of the parts of the body of the reclining person can be achieved regardless of the person's position relative to the longitudinal axis of the mattress. Furthermore, it is also possible to arrange the depressions at an angle that deviates from the 90° angle to the longitudinal axis of the mattress.

A further development of the mattress provides for having the depressions arranged in a region of the top side of the mattress core on which the parts of the body of reclining person which usually are extensively supplied with blood, such as heels, hollows of the knees, necks and/or shoulder girdles, are lying. Furthermore, the depressions can be placed in regions particularly prone to bedsores, such as heels and sacral, shoulder and/or thorax regions, as well as the back of the head. This arrangement of the illuminants in the depressions can be used effectively for bedsore prophylactics and/or to foster wound healing.

A further advantageous development of the invention provides for furnishing the depressions, preferably in part, with a translucent, preferably flexible filling material. This measure is designed to fill out the T-shaped depressions as completely as possible without causing the depressions to alter the elasticity of the mattress at all or to any appreciable degree.

According to a further advantageous development of the invention, the depressions have reflecting means by means of which the scattered light emitted by the illuminants is also directed to the top side of the mattress core. This design feature enhances the intensity of the light falling on the surface of the person's body.

Pursuant to an advantageous further development of the invention, the light intensity and/or the color of the light emitted by the illuminants can be varied by a control unit or modulated as a function of parameters. This measure improves the ability to adapt the emitted light to the needs of the reclining person. It is conceivable to have different regions of the mattress or the like assigned to illuminants which emit light at different levels of intensity and/or in different colors. For example, illuminants emitting the color green can be located in the thorax region, while having illuminants with blue light assigned to the region of the head. Depending on the demands of the specific situation, it is possible to have any number of combinations of light colors for different parts of the body.

According to a further advantageous design feature, the illuminants preferably employed are ultra-bright light-emitting diodes (LED), LED arrays, light-emitting foil made of semiconducting polymers, light-transmitting fabrics or other high-performance light sources or electroluminescent foil. Various combinations of different illuminants are possible. Different illuminants can also be assigned to different regions of the mattress. At least some illuminants, preferably all illuminants, are configured for the purpose of emitting cold light or at least essentially cold light. This makes it possible to achieve a high intensity in the light reaching the body surface of the person exposed to the light without warming the mattress core too much. At the same time, this embodiment ensures an economical production of the mattress according to the invention.

According to a preferred embodiment of the mattress, a shield, in particular an electromagnetic shield, is provided. This shield can be an integrated component of the mattress core and/or of a cover. It is also conceivable to provide at least one separate shield, which can be a cover of its own. As an alternative or additional option, it is also conceivable to assign the shield to every single illuminant, for example by having every illuminant arranged in a shield that surrounds it, preferably completely. The shield can also be formed by a sheathing which surrounds the or each illuminant. In this case, the plastic housing can be rendered conductible by means of pigments or of a conducting coating made of zinc oxide, for example, embedded in the plastic for this purpose. The respective shied ensures that the electromagnetic rays emitted by the illuminants do not reach the body of a person lying in the bed or sitting on the seating furniture. The shield can also be used to protect the respective person from other electromagnetic effect which are not, or not only, caused by the illuminants.

Finally, the mattress core is preferably constructed at least in part from a foam plastic, for example polyurethane foam or latex foam, which results in a high degree of flexibility and enhanced lying comfort for the mattress core. However, the mattress core can also be made from any other known materials.

### BRIEF DESCRIPTION OF THE DRAWINGS

A preferred exemplary embodiment of the mattress according to the invention is described in more detail below as based on the drawing, which shows:

FIG. 1 is a top view of a mattress, and

FIG. 2 is a cross-section II-II through the mattress pursuant to FIG. 1.

### DETAILED DESCRIPTION OF THE PREFERRED EMBODIMENTS

The mattress 10 shown here exhibits a conventional exterior structure. The mattress 10 is therefore suitable for use with normal beds or other reclining furniture as well as with seating furniture.

The mattress 10 essentially comprises a mattress core 17 and a cover 18. The cover 18 covers at least the top side of the mattress core 17. The cover 18 preferably encompasses the mattress core 17 completely, thus forming a wrapper of the mattress core 17. The mattress core 17 is preferably completely, or in part, composed of foam plastic. The type of foam plastic involved may be foam latex, foam polyurethane or a mixture of various foams. In the last-mentioned case the mattress core is made of a plurality of preferably horizontal layers. As an alternative, it is conceivable to have the mattress core 17 composed entirely or partially of other materials or according to different constructions, such as an internal-spring mattress.

According to the invention the mattress 10 is provided with illuminants 12 that are arranged within the contours of the mattress 10, preferably within or completely inside the interior of the mattress 10. The use of the illuminants 12 enhance the stimulus for sleeping or waking up. The illuminants 12 make such a mattress 10 also particularly suitable for bedsore prophylaxis. It has also been shown that the illuminants 12 provide the mattress 10 with beneficial properties for healing wounds.

For accommodating or housing the illuminants 12, the mattress 10 in the shown exemplary embodiment has depressions 13 in the top region of the mattress core 17. The depressions 13 proceed from the top side of the mattress core 17 and extend transversely to the longitudinal direction of the mattress 17 in a continuous manner across the entire width of the latter. Provided in the mattress 10 in the shown exemplary embodiment are a plurality of depressions—in the shown exemplary embodiment specifically five identical depressions 13. The depressions are arranged in the cover at various selected positions in relation to the longitudinal direction of the mattress 10, preferably in the heel region 14, in the region of the hollow of the knee 15 as well as in the region of the shoulder girdles 16 of a person 11 symbolically (dotted line) lying on the mattress 10. In the present case there are a number of depressions (3) arranged next to each other and spaced at a small distance from each other in the region of the knee hollows 15. But it is also conceivable to assign the depressions 13 in different arrangements or groupings relative to the top side of the mattress 10, for example at an oblique angle that deviates from the 90° angle to the longitudinal direction of the mattress 10.

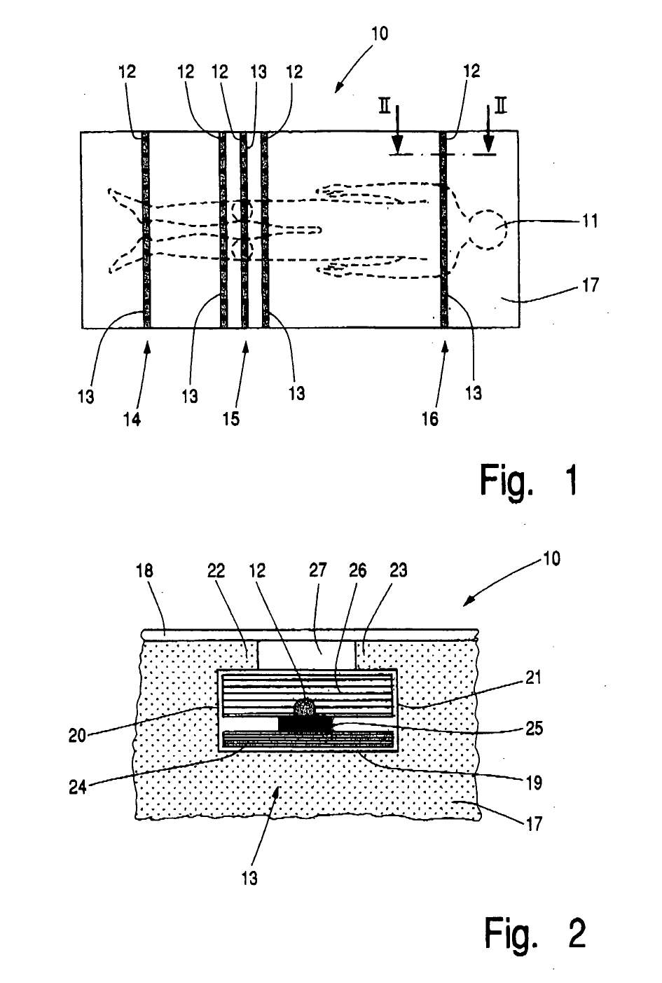

In the mattresses 10 shown in FIGS. 1 and 2 all depressions 13 have a T-shaped cross-section. The depressions 13 are arranged in the mattress core 17 as an “inverted T”. Consequently, a more narrow top region of the otherwise T-shaped depression extends from the top side of the mattress core 17. A broader, lower region of the T-shaped depression 13 thus lies under the narrow region partially covered in the interior of the mattress core 17. Accordingly, each depression 13 had a broad, flat base 19 which is bordered by two parallel, vertical side walls 20, 21. For example, the lower, broad part of the depression 13 is surrounded by the base 19 and the side walls 20, 21. The more narrow region of the depression 13 above that is bordered at the top side of the mattress core 17 by two projections 22, 23.

In the shown exemplary embodiment a uniformly configured illuminating device is arranged in every depression 13. In the following such an illuminating device will be described in more detail as based on the depression 13 shown in FIG. 2:

First of all, arranged on the base 19 of the depression 13 and between the side walls 20, 21, are preferably flexible reflecting means 24. The reflecting means 24 serve to direct the scattered light emitted by the illuminants 12 in the direction of the top side of the mattress core 17. At the same time, the reflecting means 24 can also serve as an electromagnetic shield, specifically at least as part of a shield. This constructive method increases the intensity of the light striking the body surface of the reclining person 11, thus strengthening the intended therapeutic or prophylactic effect, in particular when electromagnetic rays are held back by the shield. At the same time, this method also economizes on the energy requirement of the illuminants 12 and thus lowers the amount of heat build-up within the mattress 10.

Arranged above the reflecting means 24 is a preferably flexible circuit support 25. On the circuit support 25 are arranged a plurality of illuminants 12 with preferably equal spacing between each other. A special embodiment of light-emitting diodes (LED) are preferably employed as illuminants 12, preferably so-called ultra-bright light diodes which exhibit a high degree of efficiency and which lower the heat build-up within the mattress 10. The circuit support 25 is used as the mechanical anchoring of the illuminants 12, on one hand, and for making the electric connection and directing electric energy to the illuminants 12, on the other.

Provided above the illuminants 12 is a preferably translucent filling 26. The lens-like effect of the translucent filling 26 ensures that, among other things, the light rays emitted by the illuminant 12 are directed to the top side of the mattress core 17. The filling 26 can also be configured as an electromagnetic shield. In the shown exemplary embodiment, the filling 26 occupies only one area of the depression 13. The top, narrow region region of the T-shaped depression 13 between the two projections 22, 23 is left free by the filling 26. This constructive design makes sure that the flexibility of the mattress 10 and its ability to absorb loads, for example those exerted by a reclining person, are not impaired as a result of the integrated illuminating devices and that a reclining comfort is achieved which is comparable to that of conventional mattresses. In addition, the filling 26 achieves a padding effect, thus avoiding any “pushing through” of the filling 26 and the illuminants 12 into the cover 18 when a load is exerted on the mattress. The light emitted by the illuminant 12 first passes through the translucent filling 26, then transverses the region 27. Finally it passes through the cover 18, which at least in this zone is preferably transparent or translucent, until it strikes the body surface of the reclining person 11, where it is absorbed and triggers the desired therapeutic or prophylactic effects. Any scattered light emitted downwards by the illuminant 12 is likewise deflected upwards by the reflecting means 24 in the direction of the top side of the mattress 10.

FIG. 2 illustrates in exemplary fashion the cross-section of only one illuminant 12 exemplified by a light-emitting diode. The circuit support 25 essentially extends across the entire width of the mattress 10 and has a plurality of equally spaced illuminants 12 in a preferably linear arrangement. The same applies to the filling 26 as well as to the reflecting means 24, which also extend within the depression 13 essentially across the entire width of the mattress 10. The reflecting means 24, the filling 26 and the flexible circuit support 25 with the illuminants 12 form a functional unit and are mechanically fixed in place inside the mattress core 17 by the projections 22, 23. It would also be conceivable to secure and fix these components relative to one another and within the mattress core by means of adhesive bonding or some other connecting technology.

Instead of using the preferred ultra-bright light-emitting diodes as illuminants 12, it is also possible to employ other high-performance light sources, such as electroluminescent foil, light-transmitting fabrics, fiber optics, or light-emitting foil made of semiconducting polymers. And instead of using individual light-emitting diodes (LED) as the illuminants 12 it is also conceivable to employ so-called LED arrays in which a plurality of light-emitting diodes are combined to form a functional component assembly. All of said illuminants 12 are configured such that they radiate little or at least no appreciable heat that can be felt by a person on the mattress 10.

The example provided in FIG. 2 of the illuminant 12 positioning within the depression 13 should be regarded as only one exemplary arrangement among many. For example, it would also be possible to arrange two or more illuminants 12 parallel to one another in the form of a double row. In principle, the illuminants can be arranged in any number of geometric patterns on the circuit supporters 25.

According to a exemplary embodiment (not shown) of the invention, it is possible to recess a single depression 13 in the top side of the mattress core 17 in which a multiplicity of circuit supports 25 with illuminants, for example in the form of ultra-bright LEDs, are arranged. As another exemplary embodiment, not illustrated here, it is also possible to use a single, full-surface and preferably flexible circuit support 25 which essentially occupies the entire top side of the mattress core 17 and bears the individual illuminants 12. In this exemplary embodiment as well, the single depression 13 in the mattress 10 should be covered by a translucent fabric for the purpose of height compensation and to provide cushioning. Instead of using individually discrete LED as the illuminants 12, it is also possible to use an illuminant (not shown here) which essentially occupies the entire top surface of the mattress 10, for example in the form of a light-emitting foil made of semiconducting polymers or a luminescent foil. Optical fibers or fiberglass cloth might also be used as the illuminant 12 instead of said foils.

In a further exemplary embodiment, not shown in the drawing, illuminants 12 are arranged immediately below the cover 18 and/or between the cover 18 and the mattress core 17. Here the cover 18 is preferably made of a fabric which is light-permeable to a sufficient degree. The cover 18 can be configured as being translucent or transparent across its entire surface. As an alternative, it is also possible configure the cover 18 such that its areas of transparency are limited to those positions at which illuminants 12 are arranged. In this case, material which is less light-permeable can be used for the cover 18 in those areas where no illuminants 12 are located.

It is also possible to arrange optional or additional illuminants in a pillow and/or a bed cover. In this case, the illuminants should be positioned preferably in a pillow cover or bedspread, or the illuminants are located in a separate cover which is arranged between the pillow and pillow cover, or between the bed and the bedspread.

Furthermore, at least one shield is provided. The shield can—as mentioned above—be assigned to the reflection means 24 and/or the filling 26. As an option or in addition, it is possible to provide at least one separate shield. For example, this shield can be arranged between the mattress core 17 and the cover 18. It is also conceivable to form the mattress core 17, in total or in part, such as one or more layers of the mattress core 17, from a material which filters or shields electromagnetic radiation. Also as an option or an additional feature, the cover 18 can be configured as a shield which has properties suitable for filtering or screening out electromagnetic rays. The shield can be made from a woven or knitted fabric which does not interfere with light-permeability to any appreciable degree. For this purpose, the woven or knitted fabric can be provided with metallic fibers, such as thin wires. The wires here serve as the shield and are preferably made of silver-plated copper wire.

The illuminants 12 are arranged either within the cover 18 and/or between the cover 18 and the top side of the mattress core 17. If necessary, it can also be advantageous to cushion the illuminants 12 and the circuit support 25 on which they rest with the appropriate material, particularly if the material strength of the cover 18 is weak. A particularly advantageous solution in this exemplary embodiment can be the use of flat modular LEDs which give off cold light, for example ultra-bright LEDs. It is also conceivable to employ cold illuminants 12 of other technologies which exhibit the required geometrical dimensions. One particular advantage of this embodiment is that the cover 18 containing the illuminants 12 can be easily and subsequently put on any conventional, commercially available mattress 10.

The illuminants 12 can be configured such that they alone radiate a light of the same color. But of greater advantage is to configure the illuminants 12 such that they radiate light of different colors. For example, different parts of the body of a person lying on the mattress 10 can be provided with light having a different color. For example, the illuminants 12 in the shoulder girdle region 16 can give off a different color than the illuminants 12 in the heel region 14. In turn, the knee hollow region 15 can be irradiated with yet another color.

The delivery of different colors of light to certain parts of the body can be achieved by using basically different illuminants 12. Thus, it is also conceivable to employ illuminants 12 whose color can be altered individually by selective control mechanisms, particularly with respect to their luminous intensity.

Furthermore, a control unit (not illustrated in the figures for the purpose of clarity) is provided for regulating the illuminants 12. By means of the control unit it is first of all possible to vary the intensity of the light emitted by the illuminants 12. This makes it possible to adjust the luminous intensity to the special needs of different users.

Furthermore, in the implementation of the invention it is possible to employ illuminants 12 which can be regulated by the control unit on the basis of selected parameters with respect to properties besides the intensity of the color emitted. For example, this makes it possible to make the color of the light emitted by the illuminants 12 dependent on the part of the body to be radiated, the time of day, or on other optional parameters. For enhancing the wake-up phase, for example, it would be possible to have the illuminants 12 emit blue/green and or white light, while preferably red light would be set for the evening phase of falling asleep, if necessary only by selected illuminants, such as those in the region of the head. It is also conceivable to alter the color of light emitted only by certain illuminants 12, such as in the region of selected parts of the body, while the other parts of the body would be remain continually subjected to light of the same color or even colorless light. Of particular advantage in this case would be the use of illuminants such as multi-colored, ultra-bright LEDs. But it is also possible to use other illuminants employing alternative technologies which likewise permit variation in the color of the emitted light.

As an alternative, for example, groups of various colored LEDs can be employed as the illuminants 12. The different colored, preferably ultra-bright, LEDs can then also be turned on and off by the control unit as a function of different parameters, either individually or in groups.

According to a further exemplary embodiment of the invention (not shown), the mattress core 17 of the mattress 10 can be configured as an interior-spring mattress or as a pocket spring mattress. In this case, the illuminants 12 are preferably arranged in a foam plastic layer which covers or surrounds the interior spring or pocket spring. In addition, it is also conceivable to employ other alternative embodiments of the mattress core, such as one combining the features of interior-springs and/or latex or polyurethane plastics.

The exemplary embodiments of the invention described above merely represent an exemplary design of the mattress 10 according to the invention. For example, it would also be conceivable to apply the light-emitting configuration described above to other parts of seating or reclining furniture besides the mattress 10, in particular to a bed. For example, pillows, cushions, back cushions or bedspreads could also be configured for emitting light, specifically in the manner described in conjunction with the mattress 10. Bedclothes, pillow cases or the like could also be configured for emitting light in the manner described above. Finally, it is also conceivable to configure the mattress 10 as well as other parts of seating and reclining furniture, especially beds, for the purpose of emitting light.

A multiplicity of other exemplary embodiments of the invention are imaginable and covered by the idea of the invention, even if they are not expressly portrayed in the above description.

List of Designations

10 mattress

11 person

12 illuminant

13 depression

14 heel region

15 knee hollow region

16 shoulder girdle region

17 mattress core

18 cover

19 base

20 side wall

21 side wall

22 projection

23 projection

24 reflecting means

25 circuit support

26 filling

27 region

1. A mattress (10) having at least one mattress core (17) and a cover (18), characterized in that illuminants (12) are arranged in an area of the mattress selected from the group consisting of the mattress core (17), the cover (18), between the mattress core (17) and the cover (18), and combinations thereof.

2. The mattress (10) according to claim 1, characterized in that the mattress (10) has at its top side at least one depression (13) in which at least one illuminant (12) is arranged, it being the case that the at least one depression (13) has a preferably elongate form selected from the group consisting of an oval hollow, a grooved section, a continuous groove, and combinations thereof.

3. The mattress (10) according to claim 2, characterized in that the at least one depression (13) has an essentially T-shaped cross-section, wherein the narrow side of the cross-section points toward the top side of the mattress core (17).

4. The mattress (10) according to claim 2, characterized in that the at least one depression (13) has a length which essentially corresponds to the width of the mattress core (17) and is configured as a continuous groove extending across the entire width of the mattress.

5. The mattress (10) according to claim 2, characterized in that the at least one depression (13) is arranged preferably in such a region at the top side of the mattress core (17) on which the parts of the body which are usually intensively supplied with blood lie, such as heels, hollows of the knee, neck and upper shoulder girdles and/or regions subject to bedsores, such as heels, the sacral area, thorax area and/or the back of the head.

6. The mattress (10) according to claim 2, characterized in that at least one translucent flexible filling (26) and/or at least one preferably flexible reflecting means (24) is arranged in the at least one depression (13).

7. The mattress (10) according to claim 6, characterized in that the illuminants (12) are assigned to at least one flexible circuit support (25), with each circuit support (25) being arranged between a filling (26) and a reflecting means (24).

8. The mattress (10) according to claim 7, characterized in that the reflecting means (24) and the circuit supports (25) and/or the fillings (26) have a length which essentially corresponds to the length of the respective depression (13).

9. The mattress (10) according to claim 1, characterized by illuminants (12) which emit light of different color and/or intensity, it being possible to vary and/or modulate as a function of parameters the light intensity and/or the color of the light emitted by the illuminants (12), and if necessary it being possible to vary the duration of illumination and/or the time at which light is emitted by the illuminants (12).

10. The mattress (10) according to claim 1, characterized in that at least part of the cover (18) is configured as being translucent.

11. The mattress (10) according to claim 1, characterized in that the illuminants (12) are configured to emit essentially cold light, the illuminants (12) being selected from the group consisting of ultra-bright light-emitting diodes (LED), an array of light-emitting diodes (LED), electroluminescent foil, light-emitting foil made of semiconducting polymers, light-transmitting fabric, high-performance light sources, and a combination thereof.

12. The mattress (10) according to claim 1, characterized by at least one electromagnetic shield.

13. The mattress (10) according to claim 1, characterized in that different regions or surface sections of the mattress are associated with illuminants (12) configured for emitting light of different colors.

14. A piece of reclining furniture, in particular a bed, with a frame and at least one mattress according to claim 1 arranged thereon.

15. A piece of seating furniture and at least one set of upholstery, characterized in that the upholstery is configured in the manner of the mattress according to claim 1.
